(19) United States
(12) Patent Application Publication
(10) Pub. No.: US 2014/0207175 A1
(43) Pub. Date: Jul. 24, 2014

(54) MEDICAL DEVICE LOADING AND CARRIER TOOL

(71) Applicant: COOK MEDICAL TECHNOLOGIES LLC, Bloomington, IN (US)

(72) Inventor: Steen Aggerholm, St. Heddinge (DK)

(73) Assignee: COOK MEDICAL TECHNOLOGIES LLC, Bloomington, IN (US)

(21) Appl. No.: 13/804,271

(22) Filed: Mar. 14, 2013

(30) Foreign Application Priority Data
Jan. 18, 2013 (GB) 1300938.6

(51) Int. Cl. A61B 17/12; A61F 2/01

(52) U.S. Cl. CPC A61B 17/12131; A61F 2/01
USPC 606/200

(57) ABSTRACT

A medical device loading and carrier assembly for a medical device includes a tubular holder of transparent material so as to be see-through. The holder includes a flared proximal end for compressing a gripper element of a device carrier. The carrier can thus be activated to grip a medical device by pushing the gripper element into the tubular holder. A safety lock locks the medical device and carrier assembly in the holder to prevent inadvertent removal during storage and transportation. The holder has a distal end designed to be connected to the proximal end of a delivery sheath, such that the medical device and device carrier assembly can be slid from the holder into the sheath.

### CROSS-REFERENCE TO RELATED APPLICATIONS

This application is related to and claims the benefit of priority to GB 1300938.6, filed on Jan. 18, 2013 which is incorporated herein by reference in its entirety.

### TECHNICAL FIELD

The present invention relates to a loading tool for loading an implantable medical device into an introducer assembly. The assembly can be used with any implantable medical devices and in the preferred embodiment is designed for loading and carrying removable or repositionable medical devices such as embolization coils, vena cava filters, occlusion devices and so on. The tool can also be used for storage and transportation of implantable medical devices.

### BACKGROUND ART

Various types of implantable medical device are designed to be retrieved after a period of implantation in a patient. Examples include embolization coils, vena cava filters, occluders and the like. These types of medical device and their carrier elements are preferably provided with releasable couplings between one another. The couplings keep the medical device attached to carrier during the deployment procedure and can be used to catch the medical device for subsequent removal from a patient. The examples of assembly described below have a coupling in the form of a ball head on the medical device and resilient gripper arms on the carrier element.

Medical devices are typically attached to a carrier device, either at the time of manufacture/assembly or in the operating theatre by a clinician. Specifically, there are cases where the medical device and carrier can be loaded directly into the introducer assembly at the point of manufacture and then held therewithin during the course of endoluminal placement of the introducer assembly into the patient. In other cases, on the other hand, the medical device and carrier can only be loaded into the introducer assembly during the course of the medical procedure, for instance in cases where the introducer assembly is required for preliminary medical procedures. In such cases, the carrier sheath is left in position in the patient, the initially used medical tools removed from the assembly and then the medical device on its carrier are fed into the sheath and introduced into the patient.

In addition, in most or many instances, the medical device must be chosen on the basis of the patient's anatomy or area of the patient to be treated. In the case of embolization coils, for example, a surgeon may wish to use one or more coils of particular and different lengths. This results in having to provide a range of medical devices of different sizes for use in an operating theatre. These may be preloaded into device carriers or elements of an introducer assembly, for feeding into a patient, typically through a prepositioned carrier sheath. Medical devices which are preloaded into an introducer assembly add significant cost and volume to the assembly, contributing also to storage and handling difficulties. Moreover, there is the risk that the medical device will become disconnected from its carrier, either during storage or transportation or during the deployment operation.

The process of coupling a medical device to a carrier for implantation is not always successful, in that there is a risk that the two components do not actually couple together properly, for instance during loading of the assembly into an introducer, or that the connection becomes broken, for instance during transportation. If the two components are not properly coupled together, there is the risk that the implantable medical device will not properly deploy.

Some prior art releasable couplings for medical introducer assemblies and devices are disclosed, for example, in US-2007/0,299,461, US-2005/0154417, U.S. Pat. No. 7,942,894, U.S. Pat. No. 6,371,971 and U.S. Pat. No. 5,217,484.

### DISCLOSURE OF THE INVENTION

The present invention seeks to provide an improved implant loading and/or carrier assembly and a medical device carrier for such an assembly.

According to an aspect of the present invention, there is provided a medical device loading tool for loading a medical device into a deployment introducer; the tool including:

a tubular holder including a body portion, a distal end and a proximal end; the body portion extending between the distal and proximal ends and providing an interior to the holder for holding a medical device;

the body portion including a radial constraining device at the proximal end of the holder; wherein at least a part of the body portion is at least one of: open, transparent or translucent, for providing visual access to the interior of the holder.

The teachings herein are directed to a holder or carrier assembly which is able to hold a medical device on a carrier for loading, storage or transportation purposes, and which enables a user, such as a clinician or nurse, to inspect the state of the medical device and carrier and in particular that these two elements are properly coupled together by means of the gripper element. Should the nurse or clinician discover that these elements are not correctly coupled, they can be reloaded into the holder prior to loading into an introducer assembly. Furthermore, the holder allows a nurse or other clinician to load a medical device easily and reliably into the holder for subsequent insertion into a delivery Sheath or catheter.

Advantageously, the tubular holder is substantially entirely transparent, for example made of transparent plastics material. In other embodiments, though, the tubular holder includes a window extending preferably in the longitudinal direction of the holder and over at least the distal end of the holder, thereby to be able to see the gripper element and medical device. The window could be a slot in the holder but more preferably a closed window, which may be made of a transparent or translucent plastics material. In other embodiments, at least a proximal portion of the body portion is transparent or translucent.

The open, transparent or translucent part of the body portion may extend around a part of a circumference of the body portion.

Advantageously, the distal end of the holder includes a substantially fitting, such as a luer fitting, enabling it to be connected to the proximal end of a delivery sheath or catheter.

The radial constraining device may include a radially outwardly flared constraining portion. Preferably, at least the outwardly flared proximal end of the holder is substantially rigid.

The tool preferably includes a locking device, which may for example include one or more holes in the body portion of the holder and an engagement element provided with at least one pin passable through the or each hole.

According to another aspect of the present invention, there is provided a medical device assembly for carrying and loading a medical device into a deployment introducer; the assembly including:

an elongate medical device carrier having a proximal and a distal end;

a device gripper element provided at the distal end of the carrier;

a tubular holder within which the device carrier is disposed, the holder including a body portion delimited by a distal end and a proximal end, the distal end of the holder and the distal end of the device carrier being substantially aligned; wherein

at least a part of the body portion being at least one: of open, transparent or translucent for providing visual access to the inside of the tubular holder.

Preferably, the proximal end of the holder includes a radially outwardly flared constraining portion. Advantageously, the gripper element is resilient and laterally constrainable. The flared portion can act as a constraining device for laterally constraining the gripper element. In this regard, the gripper element may have an unconstrained width larger than the internal diameter of the tubular holder. The carrier can be loaded into the tubular holder by feeding the distal end of the carrier into the proximal end of the holder. By this action, the gripper element is progressively radially or laterally constrained as it passes further into the taper of the flared end, until it is sufficiently constrained to be able to pass into the body portion of the holder. This action will typically be carried out with a medical device located in the holder and positioned such that a coupling element of the medical device is aligned with the gripper element of the carrier and thus can be gripped thereby as the gripper element is constrained.

In the preferred embodiment, the assembly includes a locking device for locking the carrier thereto. Advantageously, the locking device includes one or more holes in the body portion of the holder and an engagement element provided with a pin or rod passable through the or each hole, the pin or pins engaging the carrier or the medical device. For this purpose, the carrier may be provided with a receiving element having one or more recesses or bores for receiving the or respective pins of the engagement element. The receiving element may be a tab fixed to the carrier.

In practice, as disclosed in detail below, the distal end of the holder is aligned into abutment with the proximal end of a catheter or sheath of an introducer assembly, whereupon the carrier, with the medical device attached thereto, can be pushed into the introducer assembly for deployment into a patient. The assembly is such that the device and its carrier can be readily passed into the introducer with little risk of disengagement of the medical device from the carrier. For this purpose, the internal diameter of the holder is preferably similar to or the same as the internal diameter of the sheath or catheter of the introducer assembly.

According to another aspect of the present invention, there is provided a combination of introducer catheter member for accommodating and releasing a medical device from a distal end thereof, a generally-tubular loading member for alignment with a proximal end of the catheter member and for supplying a medical device from the loading member into the catheter member, using a pusher member within the loading member, the pusher member being connectable to the medical device by means of an expandable gripper mechanism, which is laterally constrained to produce a retaining connection when within the loading member or the catheter member and which is openable to release the connection when released from said one end of the catheter member, wherein a proximal end of the loading member, which is opposite to the end which is aligned with the catheter member, is flared.

According to another aspect of the present invention, there is provided a method of installing an implantable medical device into a delivery catheter, comprising the steps of:

providing a medical device carrier assembly, the assembly including an elongate medical device carrier having a proximal and a distal end, a device gripper element provided at the distal end of the carrier and a tubular holder within which the device carrier is disposed, the holder including a body portion delimited by a distal end and a proximal end and a flared proximal end;

loading a medical device into the tubular device holder;

pressing the gripper element into the tubular device via the flared end of the holder to cause the gripper element to constrict laterally and thereby to grip the medical device;

aligning the distal end of the tubular holder with a proximal end of the delivery catheter; and

pushing the carrier element and the medical device into the delivery catheter.

### BRIEF DESCRIPTION OF THE DRAWINGS

Embodiments of the present invention are described below, by way of example only, with reference to the accompanying drawings, in which:

FIG. 1 is a side-sectional view of part of an introducer assembly in the course of deployment of an implantable medical device;

FIG. 2 is a side-sectional view on a slightly enlarged scale of the system of FIG. 1 after release of the medical device;

FIG. 3 is a side-sectional view of a preferred embodiment of holder of the delivery system;

FIG. 4 shows a safety lock element associated with the holder of FIG. 3;

FIG. 5 shows a medical device being loaded into the holder of FIG. 3;

FIG. 6 shows the carrier assembly and coil fitted into the holder of FIG. 3 and locked for storage and transportation purposes; and

FIG. 7 shows the subsequent transfer of the medical device from the holder into a catheter of the introducer assembly.

### DESCRIPTION OF THE PREFERRED EMBODIMENTS

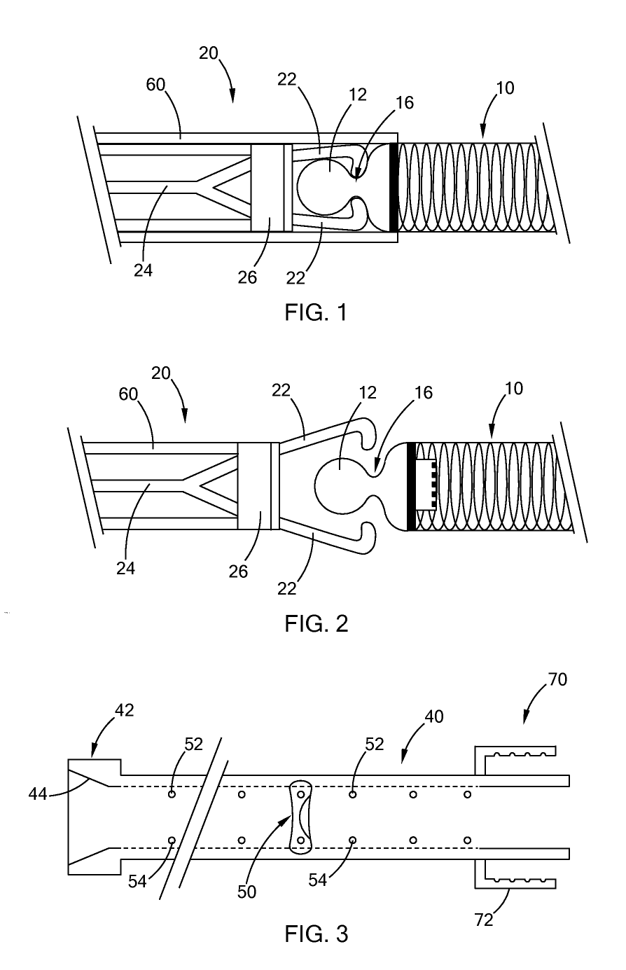

Referring to the FIG. 1, there is shown the distal end of an embodiment of introducer assembly 20 coupled to an implantable medical device 10 which in this example is an embolization coil. It is to be understood, though, that the teachings herein are not limited to any specific type of medical device as they are equally applicable to implantable medical devices in general including vena cava filters, occlusion devices and so on.

The medical device 10 is provided with a connector head 12 which in this embodiment is spherical with a narrowed neck 16. The connector 12 may be made of platinum or other inert substance. It is typically welded, for example by laser welding, to the coil or other element of the medical device 10.

The introducer assembly includes a carrier catheter or sheath 60, of generally conventional form, which at its proximal end (not visible in the drawings) is provided with a manipulation unit of generally known form for operating the elements of the carrier assembly 20 and which will include haemostatic valves, flushing ports and other known components.

Disposed within the sheath 60 is a device carrier assembly, which includes a rod, cannula or other elongate pusher 24, having attached at its distal end a gripper element formed as a pair of gripper arms 22 with inwardly extending flanges or teeth. The gripper arms 22 are made of resilient material, such as a plastics or metal. In the preferred embodiment the gripper element 22 is made from polyether ketone (PEEK). The gripper arms 22 preferably have a natural configuration in which they are spaced from one another by a distance greater than the diameter of the spherical head 12 of the device coupling member. In the configuration of FIG. 1, the gripper arms 22 are laterally or radially inwardly constrained by the inner wall of the sheath 60, thereby to close around the spherical head 12 and thereby to lock the medical device 10 thereto.

The device carrier assembly also includes a marker band or element 26 made from or which includes radiopaque material, useful in indicating the position of the distal end of the pusher 24 and thus of the proximal end of the medical device 10. This is useful in monitoring the progress of deployment of the medical device 10 from the introducer assembly. The marker band or element may be disposed around or form part of a base element of the gripper arms 22.

Referring now to FIG. 2, this shows the assembly of FIG. 1 with the carrier assembly pushed partially out of the sheath 60. As can be seen, the gripper arms 22 have moved beyond the distal end of the sheath 60 and are thus no longer constrained by the internal sheath wall, thereby able to expand radially outwardly. In so doing, the teeth or flanges move apart, allowing the spherical head 12 of the device coupling to come loose. This therefore releases the medical device 10 from the carrier.

The medical device 10 can be captured again, by locating the gripper arms 22 around the spherical head 12 and then pushing the sheath 60 forwards, which will again press the arms 22 laterally or radially inwardly, thereby to engage the head 12. This allows repositioning or removal of the medical device 10 for the patient.

In the embodiment of FIG. 2 the marker band 26′ is provided on the sheath 60 rather than the carrier assembly 20 as in the embodiment of FIG. 1.

FIG. 3 shows a side elevational view of a preferred embodiment of loading or carrier device 40. The device 40 is in the form of a tubular holder which is preferably substantially rigid and which may be made of a hard plastics material. The holder 40 is preferably transparent or translucent, although in other embodiments could be opaque with an elongate window extending longitudinally along the tube to allow visual inspection of the contents of the tube. Such a window could be an aperture in the wall of the holder, in the form of a slot, while in other embodiments could be a window with a pane of transparent or translucent material such as a plastics material. Such a window would provide visual access to the interior of the holder 40. Other embodiments could have only the proximal end of the holder 40 transparent or translucent, but this is not preferred.

The tubular holder 40 includes a distal end 70 and a tapered proximal end 42 having an internal flare wall 44, of conical form. Disposed at regular intervals along the length of the tubular holder 40 is a series of pairs of holes 52, 54 which extend into the space in the holder. The holes 52, 54 preferably have parallel bores, although these could be at a slight angle to one another, for instance at the normal to the tangent at the position of the holes.

The holes 52 and 54 need not be in pairs but this is preferred. Other embodiments could have a single hole at each longitudinal location or more than two.

The distal end 70 of the holder 40 includes, a fitting 72 which in this embodiment is a threaded fitting for coupling to a luer connector of an introducer catheter.

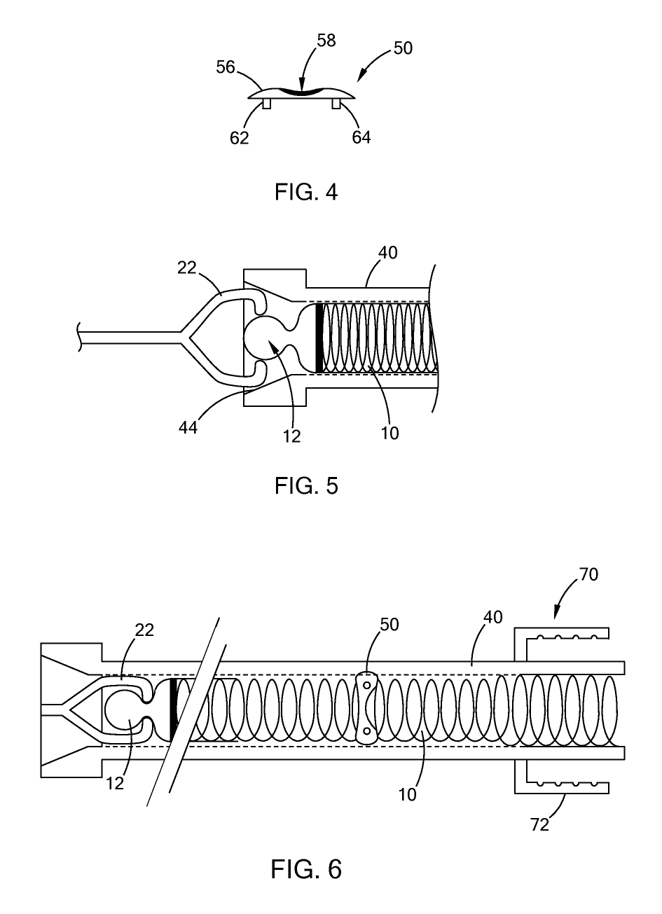

Referring now to FIG. 4, this shows a safety lock 50 for use with the assembly taught herein. The safety lock 50 includes a body element 56 which, as can be seen in FIG. 3 in particular, is of rounded rectangular form. Depending from a bottom surface of the body element 56 are first and second pins 62, 64, which has a diameter substantially the same as, or slightly smaller than, the diameter of the holes 52 and 54 of the holder 40 and spaced from one another by the same distance as the spacing of the holes 52 and 54. The top surface of the body element 56 provides a curved contour 58 for accommodating a fingertip, preferably also with transversally extending grip grooves, for ease of manipulation of the safety lock 50.

With reference to FIG. 3 also, the safety lock 50 fits onto the holder 40, at a desired set of holes 52, 54, such that its pins 62, 64 pass through their respective hole 52, 54. It is preferred that there is some resistance to the connection, which can be achieved by creating slight tension in the safety lock 50, for instance by a tight fit of the pins 62, 64 in their respective holes, by slight non-alignment of the angles of the bores of the holes 52, 54 and the respective pins 62, 64 or the like. Such tension will assist in retaining the safety lock 50 on the holder 40 until it is deliberately removed from the holder 40. It will be appreciated for this purpose that the safety lock 50 would be made of a resilient material, such as a flexible plastics material. In the alternative, an additional lock or fixator may be provided to hold the safety lock 50 in place.

The pins 62, 64 have a length significantly greater than the thickness of the wall of the tubular holder 40, such that the pins 62, 64 extend into the cavity in the holder, for reasons which will become apparent from the description which follows. The pins may have blunt ends but in other embodiments may have pointed ends.

FIG. 5 shows the proximal end 42 of the tubular holder 40, with an embolization coil 10 disposed in the holder 40 and the carrier assembly in position to be inserted into the holder 40. The gripper arms 22, which splay outwardly to their natural non-biased positions, are abutted against the tapering wall 44 of the proximal end 42, as shown. Application of force to the carrier assembly in the direction of the holder 40, that is to the right in the view of FIG. 5, will push the gripper arms 22 radially, or laterally, inwards. As can be seen in FIG. 5, the spherical head 12 of the coupling element of the coil 10 is located at the proximal end 42 of the holder 40 and such that it is within the space between the gripper arms 22. Thus, as the gripper arms 22 move down through the tapering wall 44, they will close around the spherical head 12 and thus lock the coil 10 to the carrier assembly. The gripper arms 22 will eventually be constrained sufficiently to fit within the main lumen or bore of the tubular holder 40, as shown in FIGS. 1 and 6 which follows.

It will be appreciated that the spherical head 12 will have a diameter smaller than the internal diameter of the holder 40, by an amount sufficient to accommodate the thickness of the two gripper arms 22, but its diameter is sufficiently large to retain the teeth or flanges of the gripper arms 22 held at the neck of the head 12.

Thus, the holder 40 provides a simple mechanism for securing the medical device 10 to the carrier assembly. The fact that the internal space of the holder 40 is visible, by virtue of it being transparent or having a viewing window, enables a user to verify that the coil 10 is properly coupled to the carrier element, thereby to prevent deployment of the assembly when the coil 10 has not been fixed to the carrier, as may happen with prior art arrangements. Equally, the clinician can check the coupling prior to use of the assembly in the operating theatre, in case the two elements have become separated during storage or transportation.

FIG. 6 also shows the coil 10 and carrier assembly fitted in the holder 40, as they would be provided for storage, transportation and/or loading into an introducer assembly. It is to be understood that the holder 40 may have a length sufficient to house the entire length of the medical device 10 in its pre-deployment configuration, as well at least the gripper element 22. It is not necessary for the entire length of the carrier 24 to the housed in the holder 40 and typically only the distal end of this will be so housed, with the major portion remaining outside the holder 40.

The safety lock 50 is fitted to the holder 40 and in practice its pins 62, 64 extend into the chamber of the holder 40 and, in this example, will in one embodiment engage into the space between turns of the coil 10 so as to lock these in position in the holder and as a result the entire assembly of carrier and coil. Thus, the coil 10 cannot be displaced from the holder 40, until the safety lock 50 is removed.

It is to be appreciated that the pins 62, 64 of the safety lock 50 could engage with a part of the carrier assembly rather than the medical device, for instance into suitable bores or recesses in a component of the carrier such as the marker band 26 or other support element provided on the carrier 24 and of a diameter similar to the inner diameter of the holder 40 so as to extend to proximate the holes 52, 54. The plurality of sets of holes 52, 54 provides increased choices of locking locations, which are also useful for accommodating different types and sizes of medical device 10, thus enabling a single design of holder 40 to be suitable to a large range of medical devices 10.

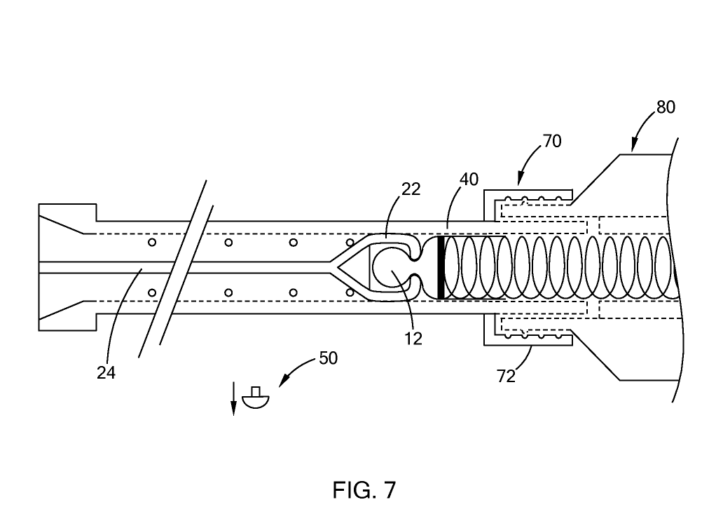

Referring now to FIG. 7, this shows the assembly in the course of loading into an introducer sheath 60. In practice, the introducer sheath 60 will have been deployed into a patient, for example by means of the well-known Seldinger technique. In other cases, the assembly of carrier and medical device could be loaded into the sheath prior to its introduction into a patient, for instance at the point of manufacture or final assembly prior to the medical procedure.

The structure and arrangement is suitable for the loading of a medical device which has been selected by the clinician in the course of a medical procedure, for instance of a coil of particular length. It is not necessary with this assembly to provide a holder or introducer assembly for each coil, which is expensive and wasteful, merely a selection of coils in suitable sterilized packaging. The chosen coil is removed from its transport packaging and then fed into the holder 40 from the distal end of the holder 40, until the connector 16 is disposed within the zone of the tapered end 42. The carrier can then be fed from the proximal end 42, by pressing the carrier into the interior of the holder 40 and thus towards the medical device 10. This action radially compresses the gripper arms 22 around the spherical head 12 and allows the distal end of the carrier 20 to pass into the cylindrical bore of the holder 40. The entire action can be seen by the clinician through the transparent wall or window of the holder 40, thereby to ensure that the medical device 10 is properly attached to the carrier 20. If this is not the case, the carrier 20 can be removed and the process repeated. There is no risk of the medical device accidentally being fed into the introducer sheath disconnected from the carrier.

The fitting 70 at the distal end of the holder 40 is then attached to a luer connector 80 at the proximal end of the sheath 60. In this regard, the internal diameter of the holder 40 is preferably the same as the internal diameter of the sheath 60 but may be smaller than the latter. Even though the internal diameter of the holder 40 could be greater than that of the sheath 60, this is not ideal as it would entail further radial compression of the contents of the holder 40 or only loose holding of these components in the holder 40.

Thus, in the preferred embodiment the tubular holder 40 has an internal diameter substantially the same as a delivery diameter of the medical device 10 held therein and of the device carrier assembly. The distal end of the holder 40 is preferably flat, that is planar in the direction normal to its axis, such that it abuts fully against the proximal end of the sheath 60.

Once the holder 40 and sheath 60 are connected together as shown in FIG. 7, the safety lock 50 is removed from the holder 40, which therefore unlocks the contents. It is only then that the contents can be moved, in this case by pushing on the proximal end of the carrier or pusher 24. This will cause the medical device 10, then the gripper element 22 and carrier 24, to slide into the sheath 60. The correspondence in internal diameters of the holder 40 and sheath 60 will ensure that the medical device 10 remains properly held in the gripper element 12 and thus fixed to the carrier 24.

The carrier pusher or rod 24 is then pushed distally to cause the medical device and the carrier to pass into the sheath 60 and then to the treatment site at the distal end of the sheath. The medical device can therefore be safely coupled to the gripper element 12 of the carrier and then fed into the delivery sheath 60 for deployment in the patient.

Once in the sheath 60, the medical device can be pushed distally (outwardly) and proximally (inwardly) in the sheath by virtue of being locked to the gripper element 12, which is advantageous during the deployment procedure. For instance, the ability to slide the medical device back and forth in the sheath 60, is useful if the medical device 10 needs to be repositioned, as well as for retrieving the medical device 10 from the patient at the end off the medical treatment. In the latter case, the gripper and carrier assembly 22, 24 can be used again, with the wall of the sheath 60 acting to compress the gripper arms radially inwardly, once they have been disposed around the spherical head 12 of the medical device 10.

The use of a spherical coupling 12 enables the medical device to rotate 360 degrees, useful during deployment and retrieval, particularly in the case of a device 10 which coils and uncoils from and to its delivery configuration.

It will be appreciated that the tubular holder 40 could be removed completely from the assembly of introducer components, by sliding this off the proximal end of the pusher 24.

It is preferred that the tubular holder 40 is substantially rigid but it is not excluded that it may be partially or entirely flexible.

The skilled person will appreciate that there are modifications to the specific embodiments describe above which fall within the broader teachings herein and the scope of the claims.

1. A medical device loading tool for loading a medical device into a deployment introducer; the tool including:
a tubular holder including a body portion, a distal end and a proximal end; the body portion extending between the distal and proximal ends and providing an interior to the holder for holding a medical device;
the body portion including a radial constraining device at the proximal end of the holder; wherein
at least a part of the body portion is at least one of: open, transparent or translucent, for providing visual access to the interior of the holder.

2. A medical device loading tool according to claim 1, wherein at least a proximal portion of the body portion is transparent or translucent.

3. A medical device loading tool according to claim 1, wherein said open, transparent or translucent part of the body portion extends around a part of a circumference of the body portion.

4. A medical device loading tool according to claim 1, wherein said open, transparent or translucent part of the body portion extends around the whole of a circumference of at least a part of the body portion.

5. A medical device loading tool according to claim 1, wherein the tubular holder is substantially entirely transparent or translucent.

6. A medical device loading tool according to claim 1, wherein the tubular holder includes a window extending in a longitudinal direction of the holder and over at least the proximal end of the holder.

7. A medical device loading tool according to claim 6, wherein the window is open or closed by a transparent or translucent pane.

8. A medical device loading tool according to claim 1, wherein the holder is substantially rigid.

9. A medical device loading tool according to claim 1, wherein the interior of the holder is substantially round in transverse cross-section.

10. A medical device loading tool according to claim 1, wherein the proximal and distal ends of the holder are open.

11. A medical device loading tool according to claim 1, wherein the distal end of the holder includes a fitting for attachment to an introducer catheter.

12. A medical device loading tool according to claim 1, wherein the fitting is a luer fitting.

13. A medical device loading tool according to claim 1, wherein the radial constraining device includes a radially outwardly flared constraining portion.

14. A medical device loading tool according to claim 13, wherein at least the outwardly flared proximal end of the holder is substantially rigid.

15. A medical device loading tool according to claim 1, including a locking device.

16. A medical device loading tool according to claim 15, wherein the locking device includes one or more holes in the body portion of the holder and an engagement element provided with at least one pin passable through the or each hole.

17. An assembly of a medical device loading tool according to claim 1 and a medical device disposed in the tubular holder.

18. An assembly according to claim 17, wherein the medical device is substantially entirely disposed in the tubular holder.

19. An assembly according to claim 17, including a carrier device disposed in the tubular holder and attached to the medical device.

20. (canceled)

21. A method of installing an implantable medical device into a delivery catheter, including the steps of:
providing a medical device carrier assembly, the assembly including an elongate medical device carrier having a proximal and a distal end, a device gripper element provided at the distal end of the carrier and a tubular holder within which the device carrier is disposed, the holder including a body portion delimited by a distal end and a proximal end and a flared proximal end;
loading a medical device into the tubular device holder;
pressing the gripper element into the tubular device via the flared end of the holder to cause the gripper element to constrict laterally and thereby to grip the medical device;
aligning the distal end of the tubular holder with a proximal end of the delivery catheter; and
pushing the carrier element and the medical device into the delivery catheter.
